(19) United States
(12) Patent Application Publication
(10) Pub. No.: US 2026/0345264 A1
(43) Pub. Date: Nov. 5, 2026

(54) Advanced Vision and Recognition Systems for Cobotic Perioperative Applications

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/428,242

(22) Filed: Dec. 21, 2025

Related U.S. Application Data
(63) Continuation-in-part of application No. 19/195,731, filed on Apr. 30, 2025.

(51) Int. Cl. A61B 34/32; B25J 9/16; G06T 7/246; G06T 7/73; G06T 17/00; G06T 19/00; G06V 10/44; G06V 10/70; G06V 20/50; G06V 40/10; G16H 40/63; A61B 34/20

(52) U.S. Cl. CPC A61B 34/32; B25J 9/1679; B25J 9/1697; G06T 7/251; G06T 7/75; G06T 17/00; G06T 19/006; G06V 10/44; G06V 10/70; G06V 20/50; G06V 40/10; G16H 40/63; A61B 2034/2065; G06T 2200/04; G06T 2207/20081; G06T 2207/30004; G06T 2210/41; G06V 2201/034

(57) ABSTRACT

A cobotic system integrates an advanced vision and recognition platform for real-time perception and adaptive control in perioperative environments. The system includes a multimodal imaging suite comprising high-resolution cameras, infrared sensors, depth sensors, and optical imaging devices. A control unit applies machine learning-based object recognition to identify instruments, anatomical structures, personnel, and procedural actions. The system generates a dynamic three-dimensional spatial map of the surgical field to support navigation, localization, and sterile field compliance. Predictive workflow analysis anticipates procedural steps and identifies deviations using real-time and historical visual data. Adaptive feedback controls adjust cobotic motion in response to changing conditions, while optional augmented reality interfaces deliver visual cues to the surgical team. The system also logs visual and analytical data for compliance, training, and continuous model refinement.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by refer

### TECHNICAL FIELD

The present invention relates generally to surgical robotics and computer vision systems, and more specifically to integrated vision and recognition platforms for cobotic use in perioperative environments.

### BACKGROUND

Modern operating rooms are dynamic environments in which surgical teams must coordinate the use of complex tools, maintain strict sterility protocols, and respond to changing procedural demands. In this context, collaborative robots, or “cobots,” have emerged as valuable assets for assisting with surgical workflows, logistics, and intraoperative support. However, effective integration of cobots into surgical settings requires systems capable of perceiving, interpreting, and responding to visual and spatial information in real time.

Existing surgical robotic systems often rely on static or manually configured settings for movement and positioning, with limited ability to autonomously interpret the evolving surgical scene. While some robotic platforms incorporate basic camera or depth sensing, these inputs are typically used for simple obstacle avoidance or teleoperation and are not fully leveraged for environmental understanding or procedural context awareness.

There is a growing need for cobotic platforms that can not only capture high-quality visual data but also perform real-time analysis to identify instruments, personnel, anatomical structures, and procedural steps. Further, maintaining situational awareness within the operating room demands the ability to generate dynamic three-dimensional maps, detect sterile field boundaries, and respond to unexpected workflow deviations. Without such capabilities, robotic systems risk compromising sterility, introducing delays, or operating suboptimally in complex and fast-changing surgical scenarios.

Additionally, current systems offer limited predictive insight. Even when imaging and tracking are available, they rarely provide real-time procedural forecasting or workflow optimization. As a result, surgical teams miss opportunities to proactively adapt to changes, improve safety, or reduce cognitive load. Likewise, many systems lack the ability to provide visual communication, such as augmented reality (AR) overlays, to inform or alert clinicians based on real-time analysis. Moreover, the absence of structured data logging and machine learning feedback loops limits continuous system improvement and regulatory compliance.

Accordingly, there exists a need for an integrated cobotic vision and recognition platform capable of high-fidelity environmental sensing, real-time object recognition, procedural forecasting, and adaptive control while also supporting safety monitoring, AR visualization, and longitudinal data capture to support clinical efficacy and operational reliability.

### SUMMARY

The present invention provides a cobotic platform incorporating an advanced vision and recognition system configured to support real-time perception, interpretation, and adaptive control within a perioperative environment. The system includes a multimodal integrated vision suite comprising high-resolution cameras, infrared sensors, depth-sensing devices, and specialized optical imaging components to capture comprehensive visual data of the surgical scene.

A control unit analyzes incoming image streams using machine learning-based object recognition algorithms to detect and track surgical instruments, anatomical structures, personnel, and procedural steps. The system further constructs and continuously updates a dynamic three-dimensional spatial map of the surgical environment, which informs cobotic navigation, positioning, and contextual awareness.

The system includes a predictive workflow analysis module that processes real-time and historical visual data to forecast upcoming procedural steps, identify deviations from expected workflows, and support surgical efficiency. A sterile field monitoring module identifies sterile boundaries and generates alerts upon detecting potential contamination risks.

The vision and recognition system is coupled with an adaptive feedback control layer that modulates the motion of the robotic platform based on the recognized environment and workflow state. Optional augmented reality (AR) interfaces provide real-time visual notifications and overlays to surgical staff. All relevant visual, spatial, and analytical data may be logged for compliance auditing, training, and iterative refinement of the machine learning models used by the system.

### BRIEF DESCRIPTION OF DRAWINGS

The foregoing and other objects, features, and advantages of the invention will become more apparent from the following detailed description, taken in conjunction with the accompanying drawings, in which:

FIG. 1 is a perspective view of a cobotic platform according to a disclosed embodiment.

FIG. 2 is a block diagram of system architecture of a cobotic platform according to a disclosed embodiment.

FIG. 3 is a flow diagram depicting the process of spatial mapping and localization.

FIG. 4 is a flow diagram depicting the integration of visual input with robotic motion planning.

FIG. 5 is a workflow diagram of the predictive procedural analysis module, showing how real-time imaging is combined with historical data to anticipate procedural steps and identify deviations.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

Embodiments of the present disclosure are described below with reference to the accompanying drawings, which illustrate an exemplary cobotic perioperative system equipped with an advanced vision and recognition platform. These examples are presented in sufficient detail to enable those skilled in the art to practice the system, but are not intended to limit the scope of the disclosure. Various modifications and alternative embodiments will be apparent to those skilled in the art.

In the drawings, like reference numerals refer to like components throughout the figures. For clarity, certain elements or features may be shown in a simplified form, exaggerated, or omitted to highlight pertinent aspects of the system.

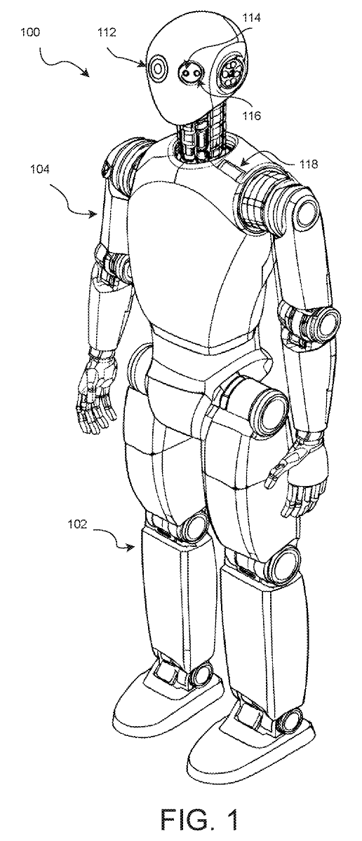

Referring to FIG. 1, a perspective view of an exemplary cobotic platform 100 is shown equipped with an integrated vision system 110. Platform 100 in this embodiment includes a mobile base 102 providing locomotion to traverse a perioperative environment (such as an operating room), and it may support a robotic arm or mast 104 for positioning sensors and interacting with surgical equipment. The platform 100 is sized and configured for use in sterile or near-sterile environments, with smooth surfaces and compliance with hospital hygiene protocols. In some embodiments, platform 100 includes safety features such as rounded edges and force-limited actuators to allow close collaboration with human staff.

The integrated vision system 110 comprises multiple imaging and sensing devices to capture rich, real-time visual data of the surgical field and its surroundings. These devices can include one or more high-resolution visible-light cameras 112 for color video imaging, infrared (IR) sensors 114 for thermal imaging and low-light visibility, and depth sensors 116 (for example, stereo cameras or LiDAR units) for three-dimensional perception. In addition, specialized optical imaging devices 118 may be provided, such as ultraviolet or fluorescence-based cameras and polarizing filters, to detect specific features like biological contaminants or tissue characteristics that are not apparent in standard imaging. Each of these sensors is operatively mounted on the platform 100 (for instance, on the arm 104 or at strategic points around the base) to achieve wide coverage of the operative scene from various angles.

In operation, the vision system 110 continuously captures synchronized streams of data from the cameras 112, IR sensors 114, and depth sensors 116 to monitor the patient, surgical staff, instruments, and environment in real time. The cobotic platform 100 can autonomously reposition itself (via base 102 and/or adjusting arm 104) to maintain optimal viewpoints or to follow key activities, ensuring critical areas remain within the field of view of sensors 110. The combination of multiple sensor modalities provides robust perception under diverse conditions, for example, IR sensor 114 can highlight temperature differences (useful for tracking personnel or recently sterilized instruments), while depth sensor 116 enables precise distance measurement and spatial awareness. Overall, the integrated vision system 110 acts as the “eyes” of the cobot, supplying comprehensive visual information that will be processed to recognize objects, map the environment, and guide the cobot’s actions as described below.

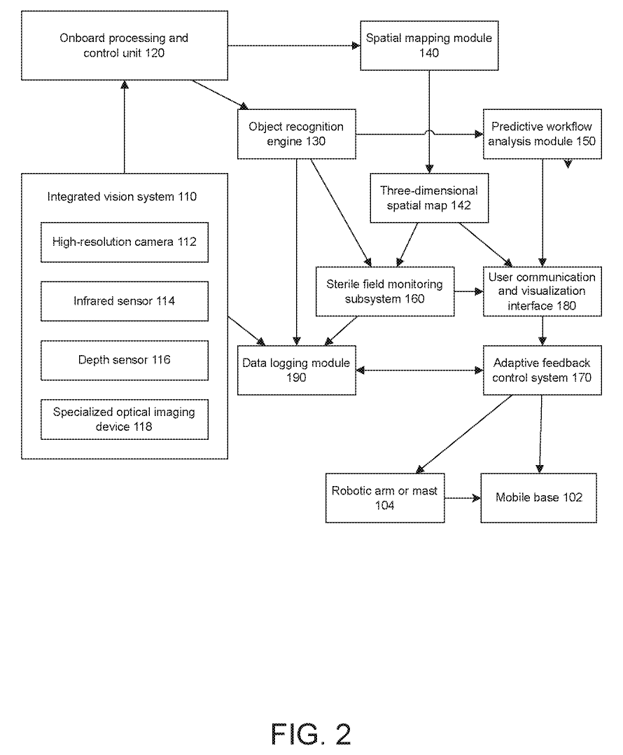

FIG. 2 is a block diagram illustrating the system architecture of the vision-enabled cobot. Raw data streams from the integrated vision system 110 (including all sensors 112, 114, 116, 118) are transmitted to an onboard processing and control unit 120. The processing unit 120 may be a dedicated computing module (for example, one or more high-performance processors or GPUs) configured to analyze images and sensor data in real time. Within unit 120, a real-time object recognition engine 130 processes incoming images to identify and classify objects of interest (such as surgical instruments, personnel, or anatomical features), and a spatial mapping module 140 concurrently fuses depth data and visual cues to construct a dynamic three-dimensional model 142 of the environment. The object recognition engine 130 and mapping module 140 operate in parallel and share data: for example, the positions of recognized objects from engine 130 can be overlaid onto the 3D map 142 generated by module 140, yielding a comprehensive situational awareness of the cobot’s surroundings.

Higher-level analysis components make use of the outputs from recognition engine 130 and mapping module 140. For instance, a predictive workflow analysis module 150 monitors the sequence of recognized actions and objects over time (as provided by engine 130), comparing these patterns to historical procedural data to forecast upcoming steps in the surgical procedure. Additionally, a sterile field monitoring subsystem 160 utilizes the spatial map 142 and object location data to enforce surgical sterility protocols: it defines boundaries of the sterile field within the map and checks whether any tracked object or person crosses these boundaries inappropriately. Module 160 can automatically detect potential contamination risks (e.g., a non-sterile object being introduced into a sterile zone or a lapse in proper gown/glove usage by personnel) and will flag these events for the system to address or to alert the surgical team.

The system architecture further integrates an adaptive feedback control system 170 that governs the cobot’s locomotion and any robotic manipulator functions. Control system 170 receives continuous input from the mapping module 140 and recognition engine 130, enabling the cobot to react in real time to changes. For example, if the object recognition 130 identifies an obstacle or a new surgical instrument drop location, and mapping 140 pinpoints its position, the control system 170 can adjust the path or pose of the cobot to avoid a collision or to improve sensor viewpoint. Thus, control 170 closes the loop by translating perceptual insights into physical actions of the base 102 and arm 104.

The system includes a user communication and visualization interface 180, and a data logging module 190. The interface 180 delivers information to users (such as surgeons or support staff) in real time, potentially through augmented reality overlays or display screens. Concurrently, the data logging module 190 records all relevant sensor data, recognition events, system decisions, and alerts for post-operative review, compliance documentation, and machine learning model improvement. Together, the components depicted in FIG. 2 form an interconnected architecture that enables the cobot to perceive, understand, and interact with the surgical environment intelligently and safely.

Referring still to FIG. 2, the system architecture further supports user-facing communication, alerting, and persistent data capture based on outputs generated by the processing, analysis, and control components of the cobotic system. As illustrated, information derived from the object recognition engine 130, spatial mapping module 140, predictive workflow analysis module 150, sterile field monitoring subsystem 160, and adaptive control system 170 may be selectively routed to downstream components responsible for visualization and data persistence.

In some embodiments, when the processing and analysis components identify a condition of interest, such as a predicted forthcoming procedural step, a detected anomaly or deviation in workflow, proximity between the cobotic platform 100 and surgical staff, or a potential breach of a predefined sterile field boundary, the system generates contextual outputs reflecting the identified condition. Such contextual outputs may include safety alerts, workflow-related notifications, or visual cues derived from the current state of the system and its representation of the surgical environment.

The generated contextual outputs may be communicated to users via the user communication and visualization interface 180. In some embodiments, the interface 180 includes an augmented reality interface configured to present spatially registered visual overlays aligned with a live view of the surgical field. For example, the interface 180 may highlight a sterile field boundary monitored by the sterile field monitoring subsystem 160, emphasize the location of a tracked instrument or anatomical structure identified by the object recognition engine 130, or present a visual cue corresponding to a predicted or anomalous procedural state determined by the predictive workflow analysis module 150.

In parallel with user communication, system outputs and associated contextual data may be recorded by the data logging module 190. The logged data may include time-stamped sensor inputs from the integrated vision system 110, recognition results generated by the object recognition engine 130, predictive outputs and deviation classifications produced by the predictive workflow analysis module 150, alerts issued by the sterile field monitoring subsystem 160, and control actions executed by the adaptive control system 170. This persistent record supports post-procedure review, compliance auditing, personnel training, and refinement of machine learning models used by the recognition and predictive components of the system.

By integrating user communication and data persistence directly within the system architecture of FIG. 2, the cobotic system 100 can enable alerts, visualizations, and recorded data that are derived from and remain consistent with the same processing and control components that govern perception, prediction, and motion, thereby improving situational awareness, safety, and system accountability without requiring a separate downstream output figure.

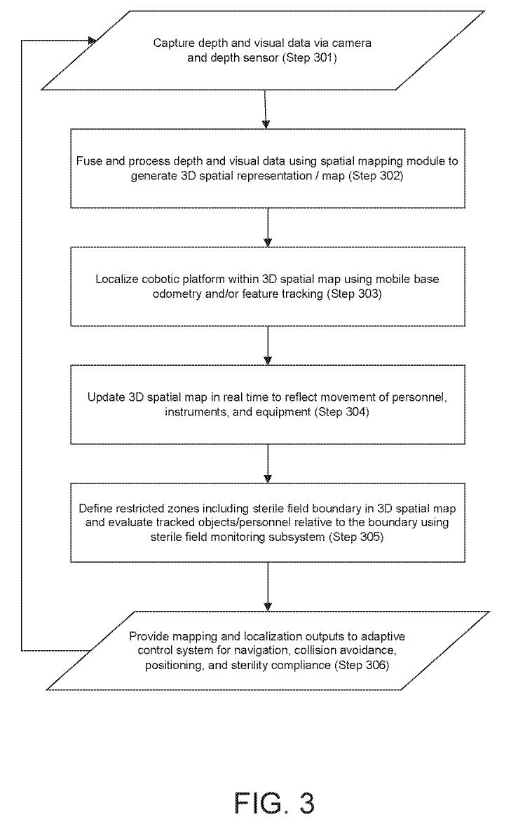

Referring now to FIG. 3, a schematic diagram is shown illustrating a spatial mapping and localization process performed by the cobotic system to generate and maintain a dynamic three-dimensional representation of a perioperative environment. The diagram represents a sequence of method steps by which visual and depth data are processed to determine spatial relationships among the cobotic platform, surgical personnel, instruments, and environmental structures.

At Step 301, the integrated vision system captures depth data and visual image data from the surgical environment using one or more depth sensors and cameras mounted to the cobotic platform. The captured data reflects surfaces, objects, and structural features within the operating room, including the operating table, surrounding equipment, personnel, and the cobotic platform itself.

At Step 302, the captured depth and visual data are fused and processed by a spatial mapping module to generate a three-dimensional spatial representation of the surgical environment. The spatial representation may be constructed as a point cloud, mesh, or volumetric model in which surfaces and obstacles are identified and spatially resolved.

At Step 303, the cobotic system performs localization by determining a position and orientation of the cobotic platform within the generated three-dimensional spatial map. Localization may be based on feature tracking, fiducial markers, odometry from a mobile base, or a combination thereof, allowing the system to continuously track its own movement relative to the mapped environment.

At Step 304, the spatial mapping module updates the three-dimensional spatial map in real time to reflect changes in the environment, including movement of personnel, relocation of surgical instruments, or repositioning of equipment. As objects move or are introduced into the environment, their updated positions are incorporated into the spatial representation.

At Step 305, one or more restricted or context-sensitive zones are defined within the three-dimensional spatial map, including a predefined sterile field boundary associated with a surgical site. The system evaluates tracked objects and personnel relative to the defined boundaries to determine whether a potential breach or encroachment has occurred or is likely to occur.

At Step 306, spatial mapping and localization outputs are provided to downstream system components, including adaptive control logic and safety monitoring subsystems, to support collision avoidance, safe navigation, and sterility compliance. Following Step 306, the process returns to Step 301 to continue continuous spatial mapping and localization throughout the perioperative procedure.

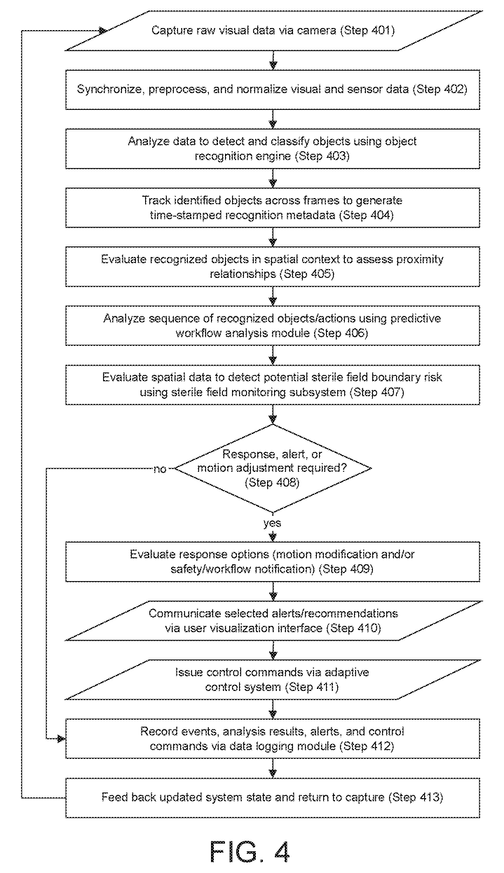

Referring now to FIG. 4, a flow diagram is shown illustrating a continuous vision-to-control process executed by the cobotic system during operation within a perioperative environment. The flow diagram represents a sequence of method steps that are repeatedly performed to integrate real-time visual perception with adaptive robotic motion planning and control.

Referring now to FIG. 4, a flow diagram is shown illustrating a multi-stage, closed-loop process by which visual sensing, object recognition, workflow analysis, safety monitoring, user communication, and adaptive robotic control are coordinated during perioperative operation of the cobotic system. The flow diagram depicts a sequence of discrete method steps that are executed iteratively to enable real-time situational awareness, proactive workflow support, and safe collaborative motion within a dynamic surgical environment.

At Step 401, the integrated vision system captures raw visual data from the surgical environment using one or more cameras 112, optionally in combination with infrared and depth sensors, to obtain real-time views of the operating room.

At Step 402, the captured visual and sensor data is synchronized, preprocessed, and normalized to account for lighting conditions, sensor alignment, and temporal consistency across multiple data streams.

At Step 403, the preprocessed data is analyzed by the object recognition engine to detect and classify objects present in the surgical environment, including surgical instruments, personnel, anatomical structures, and environmental features.

At Step 404, the system tracks identified objects across successive frames to determine motion trajectories, interaction events, and changes in object state, generating time-stamped recognition metadata.

At Step 405, the recognized objects and tracking data are evaluated in a spatial context to assess proximity relationships between the cobotic platform, personnel, instruments, and anatomical structures.

At Step 406, the predictive workflow analysis module 150 analyzes the sequence of recognized objects and actions in view of historical surgical data or predefined procedural models to determine a current procedural context, predict a forthcoming procedural step, or identify a deviation from an expected workflow.

At Step 407, the sterile field monitoring subsystem 160 evaluates the spatial data to determine whether any tracked object or person has crossed, or is likely to cross, a predefined sterile field boundary, thereby identifying a potential contamination risk.

At Step 408, the system aggregates outputs from the object recognition, workflow prediction, and sterile field monitoring processes to determine whether a response, alert, or motion adjustment is required.

At Step 409, response options are evaluated, including selection of a motion modification, generation of a safety alert, issuance of a workflow-related notification, or a combination thereof.

At Step 410, selected alerts, recommendations, or contextual information are communicated to surgical staff via the augmented reality or user visualization interface 180, which may overlay visual cues, highlight relevant objects, or indicate predicted procedural steps within a live view of the surgical field.

At Step 411, the adaptive control system 170 generates and issues control commands to the cobotic platform to implement the determined response, including adjusting movement of the mobile base, repositioning a robotic arm or sensor, modifying speed or applied force, or altering camera orientation.

At Step 412, operational data, recognized events, analysis results, issued alerts, and control commands are recorded by the data logging module 190 for compliance auditing, training, and system learning purposes.

At Step 413, updated system state information resulting from execution of the control commands is fed back to the integrated vision system, and the process returns to (Step 401) to continue continuous monitoring, analysis, and adaptive control.

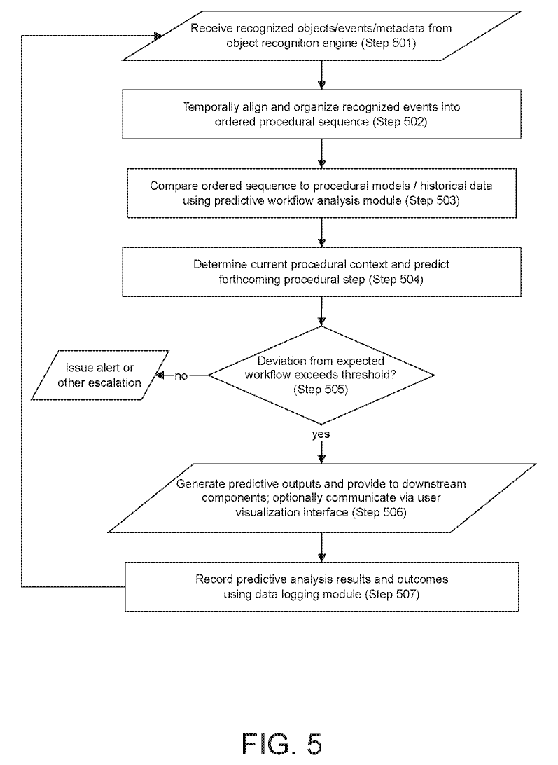

Referring now to FIG. 5, a workflow diagram is shown illustrating a predictive procedural analysis process executed by the predictive workflow analysis module during a perioperative procedure. The workflow diagram represents a sequence of method steps by which real-time visual recognition data is combined with historical procedural data to determine a current procedural context, predict forthcoming procedural steps, and detect deviations from an expected surgical workflow.

At Step 501, the predictive workflow analysis module receives, as input, a continuous stream of recognized objects, events, and contextual metadata generated by the object recognition engine. The received data may include identified surgical instruments, detected personnel actions, temporal ordering of procedural events, and spatial relationships within the surgical environment.

At Step 502, the received real-time recognition data is temporally aligned and organized into an ordered sequence representing recent procedural activity. This step may include filtering, weighting, or segmenting recognized events to emphasize clinically relevant actions and to reduce noise or transient detections.

At Step 503, the predictive workflow analysis module compares the organized sequence of recognized events against one or more stored procedural models, historical surgical datasets, or predefined workflow templates corresponding to a particular surgical procedure. The comparison may be performed using a machine learning model, probabilistic model, or rule-based analysis to evaluate similarity to known procedural patterns.

At Step 504, based on the comparison, the predictive workflow analysis module determines a current procedural context and generates a prediction of at least one forthcoming procedural step, including an estimated timing or confidence level associated with the prediction.

At Step 505, the module evaluates whether the observed sequence of events deviates from an expected workflow pattern beyond a predetermined threshold, thereby identifying a potential anomaly, delay, or deviation in the procedure. When such an anomaly, delay, or deviation is identified, the predictive workflow analysis module classifies the deviation based on one or more factors including severity, duration, confidence level, and clinical relevance. The classified deviation is then forwarded for downstream handling, which may include generation of a workflow-related alert, issuance of a recommendation, adjustment of system behavior, or escalation to user notification, as further described with respect to subsequent steps. In some embodiments, the evaluation further distinguishes between transient deviations that self-resolve and sustained deviations that warrant proactive intervention.

At Step 506, predictive outputs, including predicted next steps, detected deviations, or workflow-related recommendations, are generated and made available to downstream system components. In some embodiments, the predictive outputs are communicated to surgical staff via a user visualization or augmented reality interface, enabling proactive preparation or corrective action without interrupting the procedure.

At Step 507, predictive analysis results, together with corresponding real-time observations and eventual procedural outcomes, are recorded to a data storage or logging module. The recorded data may subsequently be used to refine procedural models, retrain machine learning algorithms, or improve predictive accuracy in future procedures. Following completion of Step 507, the process returns to Step 501 to continue ongoing workflow prediction throughout the procedure.

It will be appreciated that the foregoing embodiments and workflows described with reference to FIGS. 1–5 are presented for example and not limitation. Various modifications in form, arrangement, and detail can be made without departing from the scope of the cobotic vision and recognition system. For instance, features illustrated in one figure may be combined with those of another, alternative sensor types may be used in the vision system 110, and the system may be adapted to different surgical procedures or environments. The scope of the invention, accordingly, is defined by the claims and equivalents thereof rather than by the specific examples given above.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A cobotic system for perioperative applications, comprising:
an integrated vision system comprising a plurality of sensors configured to capture multimodal image data of a surgical environment;
a robotic apparatus configured for collaborative operation within the surgical environment; and
a control unit operatively coupled to the integrated vision system and the robotic apparatus, the control unit being configured to:
analyze, in real time, the captured image data using a machine learning algorithm to recognize objects and features within the surgical environment;
generate a dynamic three-dimensional spatial map of the surgical environment based on the captured image data; and
provide adaptive motion control commands to the robotic apparatus in response to the recognized objects and the spatial map.

2. The system of claim 1, wherein the integrated vision system comprises at least one high-resolution camera, at least one infrared sensor, at least one depth sensor, and at least one optical imaging device to capture different imaging modalities of the surgical environment.

3. The system of claim 1, wherein the control unit’s machine learning algorithm is trained on surgical data to identify a plurality of surgical instruments, anatomical features of a patient, and personnel within the surgical environment from the captured image data in real time.

4. The system of claim 1, wherein the control unit is further configured to determine a current stage of a surgical procedure by analyzing patterns of recognized objects and actions over time against a predefined surgical workflow model.

5. The system of claim 1, wherein the control unit constructs and continuously updates the three-dimensional spatial map of the surgical environment to track locations of the recognized objects and to provide real-time spatial localization of the robotic apparatus and surgical instruments.

6. The system of claim 1, wherein the control unit is configured to monitor a predefined sterile field boundary within the surgical environment and to generate an alert if a breach of the sterile field is detected.

7. The system of claim 1, wherein the control unit is configured to dynamically adjust at least one of a movement path, speed, or applied force of the robotic apparatus in response to the proximity of surgical staff or delicate anatomical structures identified by the integrated vision system, thereby maintaining safe collaboration between the robotic apparatus and personnel.

8. The system of claim 1, further comprising a user interface configured to provide real-time feedback to an operator, wherein the feedback includes information about the recognized objects or system status conveyed via at least one of a visual display or an audio output.

9. The system of claim 8, wherein the visual display comprises an augmented reality interface that overlays the information onto a live view of the surgical field to highlight recognized objects and provide contextual cues during the procedure.

10. The system of claim 1, further comprising a data logging module configured to record the captured image data, recognized events, and control commands, wherein the recorded data is usable for at least one of surgical audit, personnel training, or machine learning model improvement.

11. A cobotic system for perioperative applications, comprising:
an imaging system capturing real-time images of a surgical procedure;
a robotic apparatus operable within a surgical environment;
a control unit configured to analyze the real-time images to recognize a current surgical context and to predict a forthcoming procedural step or detect a deviation from an expected workflow by comparing the real-time images with historical surgical data; and
an augmented reality interface configured to present to a user a real-time visualization overlay of information related to the recognized context, including indications of any predicted next step or detected workflow deviation.

12. The system of claim 11, wherein the control unit’s predictive workflow analysis utilizes a machine learning model trained on historical surgical procedures to model expected sequences of steps, such that the control unit updates its predictions based on both current image data and stored procedural patterns.

13. The system of claim 11, wherein the augmented reality interface comprises a head-mounted display or screen that overlays indicators onto a live surgical view, the indicators including at least one of identified surgical instruments, anatomical landmarks, or a visual cue highlighting an upcoming procedural step.

14. The system of claim 11, wherein the control unit is configured to detect a deviation from the expected surgical workflow in real time and, in response, to generate an alert or recommendation via the augmented reality interface to notify surgical staff of the deviation and suggest corrective action.

15. The system of claim 11, wherein the control unit is configured to automatically adjust one or more operating parameters of the robotic apparatus or to suggest a repositioning of the robotic apparatus in anticipation of the predicted next step, thereby preparing the cobotic system to assist with the upcoming action.

16. The system of claim 11, further comprising a data storage module that records predictive analysis data alongside actual surgical outcomes, wherein feedback from the recorded outcomes is used to refine the predictive workflow analysis module for improved accuracy in future procedures.

17. A method for assisting a perioperative procedure using a collaborative robotic system, the method comprising:
capturing multimodal image data from a surgical environment in real time via an integrated vision system mounted to or associated with a collaborative robotic platform;
analyzing the captured image data using a machine learning algorithm to recognize objects in the surgical environment, including surgical instruments, anatomical features, and personnel positions;
generating and continuously updating a three-dimensional spatial map of the surgical environment based on the analyzed image data;
detecting a breach of a predefined sterile field boundary in the surgical environment from the spatial map and issuing a safety alert in response to the detected breach; and
adjusting a movement of the collaborative robotic platform or an attached robotic instrument in real time based on the recognized objects and the spatial map to avoid collisions or interference with ongoing surgical activities.

18. The method of claim 17, further comprising analyzing a sequence of the captured images in conjunction with historical surgical data to predict a next action in the procedure or to detect a deviation from an expected procedural workflow.

19. The method of claim 17, further comprising presenting, on an augmented reality display, a real-time overlay that highlights at least one recognized object or provides a cue indicating the predicted next step of the procedure to a user in the operating room.

20. The method of claim 17, further comprising logging the captured image data, recognized events, or control actions to a data repository for at least one of surgical audit, training purposes, or improving a machine learning model associated with the collaborative robotic system.
